(19) United States
(12) Patent Application Publication
(10) Pub. No.: US 2024/0117290 A1
(43) Pub. Date: Apr. 11, 2024

(54) APPARATUS, SYSTEM, AND METHOD FOR BIOPROCESSING UTILIZING A ROCKER BIOREACTOR

(71) Applicant: GLOBAL LIFE SCIENCES SOLUTIONS USA LLC, Marlborough, MA (US)

(72) Inventors: MOJTABA PARVIZI, NEEDHAM, MA (US); CRAIG MIZZONI, FOXBOROUGH, MA (US); MACKENZIE RYAN, MARLBOROUGH, MA (US)

(73) Assignee: GLOBAL LIFE SCIENCES SOLUTIONS USA LLC, Marlborough, MA (US)

(21) Appl. No.: 17/959,539

(22) Filed: Oct. 4, 2022

(51) Int. Cl. C12M 3/06; B01F 31/23; B01F 35/513; C12M 1/00

(52) U.S. Cl. CPC C12M 27/16; B01F 31/23; B01F 35/513; C12M 23/14; C12M 23/50

(57) ABSTRACT

In an embodiment, a bioprocessing method includes securing a vessel containing a fluid to a platform capable of pivoting about a first axis, heating the fluid in the vessel while the platform is in a static, substantially upright position to facilitate a first bioprocessing procedure within the vessel. The method further includes pivoting the platform and vessel about the first axis to create a rocking motion to facilitate a second bioprocessing procedure within the vessel.

### BACKGROUND

### Technical Field

Embodiments of the invention relate generally to bioprocessing apparatus, systems, and methods, and, more particularly, to increasing the functionality of a rocker bioreactor so that it may perform multiple bioprocessing procedures.

### Discussion of Art

Bioreactors are often employed to carry out biochemical and/or biological processes and/or manipulate liquids and other products of such processes. Such bioreactors often include flexible or collapsible single-use disposable bags that are supported by an outer rigid structure. These “single-use” bioreactors include stirred tank reactors, in which a sterile disposable bag and agitator are housed within a rigid tank, and rocker bioreactors in which a single-use bag is secured to a rocking platform.

In rocker bioreactors, a disposable bag is secured to a tray which, in turn, is attached to a motorized rocking device. The rocking device causes the tray to pivot back and forth about an axis/pivot point. The motion created by the rocking device induces waves in cell culture media within the bag. The waves provide mixing and gas to liquid transfer of oxygen supplied to the bag, resulting in an ideal environment for cellular growth. To monitor and facilitate cellular growth, rocker bioreactors include, among other features, dissolved oxygen (DO) and pH optical sensors, often removably secured to the disposable bag, as well as heating elements and temperature sensors, which may be incorporated into the tray or other components of the rocking device.

In certain known rocker bioreactors, the tray may be tilted about a second axis/pivot point toward a user into a substantially upright sampling/vessel installation position. This upright position facilitates removal or installation of a bag onto the tray, as well as sampling, harvesting, and drainage of the bag. Typically, however, in the upright position the reactor is not fully operational, e.g., in addition to not rocking, media will be gravity driven to the bottom of the bag and tray where there are no temperature sensors. Moreover, tray heating elements are not functional in the upright position.

In addition, known rocker bioreactors are not suitable for working with media containing low starting cell numbers/low seeding volumes. As a result, known rocker bioreactors are suitable primarily for cellular growth, e.g., cell expansion. Other processes, such as, for example, activation of T-cells in chimeric antigen receptor (CAR) T cell therapy manufacturing, which typically involve low seeding volumes, must be accomplished using additional equipment.

In view of the above, there is a need for a rocker bioreactor that can perform multiple bioprocesses including, but not limited to, cell therapy manufacturing steps that involve low starting cell seeding volumes.

### BRIEF DESCRIPTION

Certain embodiments commensurate in scope with the originally claimed subject matter are summarized below. These embodiments are not intended to limit the scope of the claimed subject matter, but rather these embodiments are intended only to provide a summary of the possible embodiments. Indeed, the disclosure may encompass a variety of forms that may be like or different from the embodiments set forth below.

In an embodiment, a bioprocessing method includes securing a vessel containing a fluid to a platform capable of pivoting about a first axis, heating the fluid in the vessel while the platform is in a static, substantially upright position to facilitate a first bioprocessing procedure within the vessel. The method further includes pivoting the platform and vessel about the first axis to create a rocking motion to facilitate a second bioprocessing procedure within the vessel.

In another embodiment, a system for processing fluid in a vessel includes a tray for removable attachment to a platform that is capable of pivoting about a first axis, the tray having a first end and a second end. The system further includes a first heating element and a first temperature sensor that are proximate to the first end of the tray and an attachment mechanism for securing the vessel to the tray. The system further includes a controller configured for heating the fluid in the vessel while the platform is in a static, substantially upright position to facilitate a first bioprocessing procedure within the vessel and for pivoting the platform and vessel about the first axis to create a rocking motion to facilitate a second bioprocessing procedure within the vessel.

In yet another embodiment, an apparatus for heating fluid in a vessel includes a first heating element and a first temperature sensor and a mechanism for attaching the apparatus to a tray that is capable of pivoting about a first axis, the tray having a first end and a second end. The apparatus being configured for selective attachment at a location proximate to the first end of the tray and for heating the fluid in the vessel while the tray is in a static, substantially upright position during a first bioprocessing procedure within the vessel, and for heating the fluid while the tray is pivoting about the first axis during a second bioprocessing procedure.

### DRAWINGS

The present invention will be better understood from reading the following description of non-limiting embodiments, with reference to the attached drawings, wherein below:

FIG. 1 is a perspective view of a rocker bioreactor system suitable for use with embodiments of the present invention;

FIG. 2 is a side view of the rocker bioreactor system of FIG. 1 depicting a substantially upright tray position suitable for use with embodiments of the present invention;

FIG. 3 is a partially exploded rear perspective view of the rocker bioreactor system of FIG. 2;

FIG. 4 is a perspective view of the rocker bioreactor of FIG. 1 with the lid and tray removed so that its existing temperature sensors are viewable.

FIG. 5 is a perspective view of the rocker bioreactor of FIG. 1 with the lid and tray removed depicting an intermediate plate in a substantially upright position.

FIG. 6 is a front view of an existing tray configured for use with the rocker bioreactor of FIG. 1;

FIG. 7 is a front view of a rocker bioreactor tray according to an embodiment of the invention;

FIG. 8 is a perspective view of a rocker bioreactor with the lid and tray removed depicting an intermediate plate according to an embodiment of the invention.

FIG. 9 is a front view of a rocker bioreactor tray according to an alternative embodiment of the present invention; and

FIG. 10 is an illustration of use of a bioreactor with vessel in a substantially upright position according to embodiments of the present invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and/or “semi-rigid” are used herein interchangeably to describe structures that are “non-collapsible,” that is to say structures that do not fold, collapse, or otherwise deform under normal forces to substantially reduce their elongate dimension.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single-use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid container or vessel used, for example, as a bioreactor or mixer for the contents within. While embodiments of the present invention are described as for use with bioprocessing bags, including but not limited to bioreactor bags and mixer bags, embodiments may also be configured for use with other bags or vessels.

As used herein, “platform” refers to a rocker bioreactor surface that is configured to receive and support a vessel/bag during a bioprocessing procedure. This term includes, but is not limited to, bioreactor trays. While shown and described in association with specific rocker bioreactor systems that include, for example, trays that are affixed or mounted to rocking platforms and intermediate plates, the invention is not limited to such systems.

Embodiments may be used to perform a variety of bioprocesses in a rocker bioreactor and are not limited to cellular activation and expansion. Certain embodiments may be broadly used in bioprocessing and biochemical environments, and potentially in non-biological/biochemical contexts. Likewise, while embodiments are described and depicted with respect to specific rocker bioreactor systems, embodiments may potentially be utilized with other types of reactors/mixers, particularly those in which a vessel/bag can be secured in a substantially upright position or where supplemental heating and/or temperature sensing is desired.

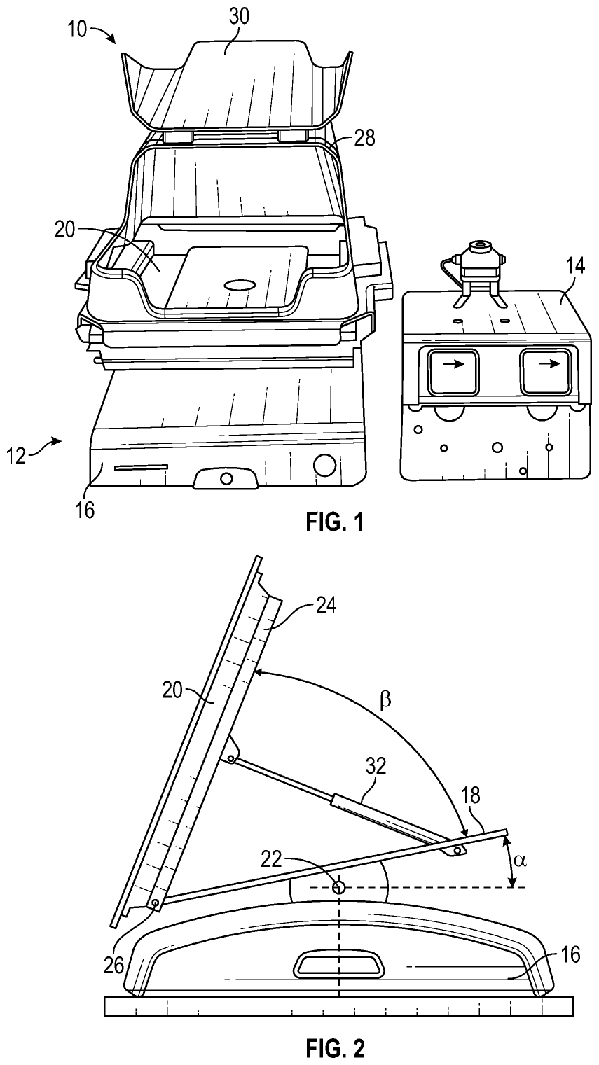

Referring to FIG. 1, an exemplary rocker bioreactor system 10 suitable for use with embodiments of the invention is depicted. As shown, the system 10 includes a bioreactor 12 which is connected to one or more peristaltic pumps 14. The bioreactor 12 is also operatively connected to a controller, e.g., a local or remote computer that provides process/protocol monitoring and the like via a wired or wireless connection (not shown).

The bioreactor 12 includes a removable tray 20, which is configured to selectively receive and support a vessel/bag. In embodiments, the tray 20 includes a removable lid 28. The lid 28 features a hinged door 30 that may be raised to gain access to the tray 20, and any vessel secured thereto.

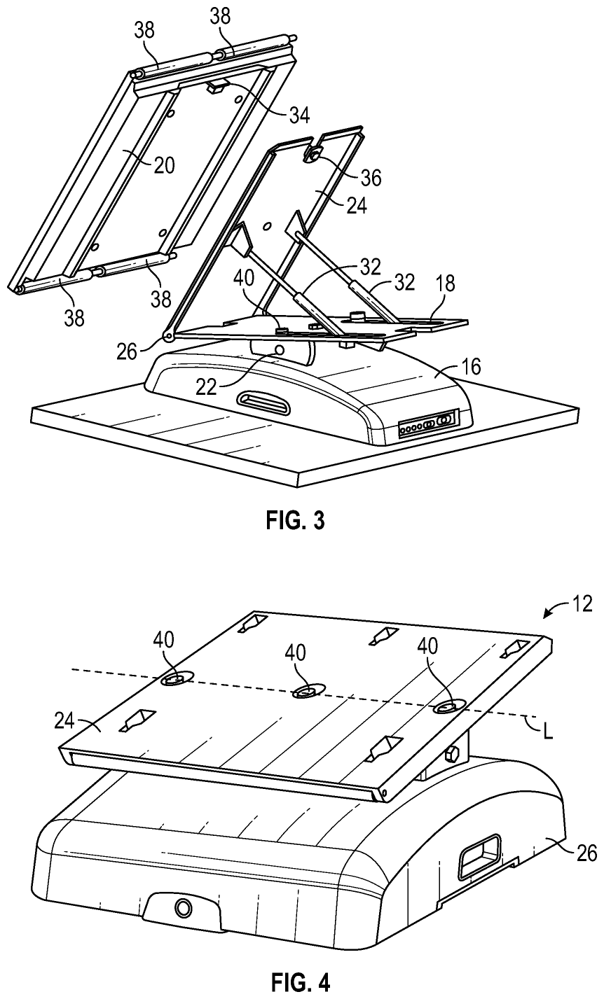

Referring now to FIGS. 2 and 3, the bioreactor 12 includes a base 16 that is connected to a rocking platform 18, which, in turn, is connected to the tray 20. The base 16 houses a pivoting mechanism, for example, a motor, which enables the rocking platform 18 to pivot back and forth about a first axis 22. While rocking, the platform 18 forms an angle α to horizontal at the end point of its upward path, and the rear portion of the rocking platform 18 moves back and forth from an angle of −α to +α. In embodiments, angle α may be varied by a user and is in a range of about 2° to about 12° As will be appreciated, however, embodiments of the invention are suitable for use with reactor platforms that move at angles outside of this range.

The rocking platform 18 is, in turn, pivotally connected to an intermediate plate 24 which may be tilted toward a user about a second axis 26 until the intermediate plate 24 is substantially upright. The intermediate plate 24 (and any tray 20 secured thereto) pivots about the second axis 26 to angle β, which is about 60° from the rocking platform 18, which may also be tilted forward at angle α. In total, the rocking platform 18 and tray 20 form an angle α+β of between about 60-90° from horizontal, and, in a particular embodiment, about 72° from horizontal. Thus, in embodiments, the “substantially upright” refers to a platform/tray that is approximately 60-90° from horizontal.

Once the vessel (e.g., bioreactor bag) has been attached or sampling has occurred, the rocking platform 18 may be returned to a substantially horizontal position via the second axis 26. The rocking platform may then pivot about the first axis 22 creating a rocking motion to facilitate bioprocessing.

Referring now to FIGS. 2 and 3, movement of the intermediate plate 24 relative to the rocking platform 18 may be guided/controlled via one or more gas dampers 32. In embodiments, the dampers 32 hold the intermediate plate 24 and tray 20 (and any vessel present) in the substantially upright position while also allowing the intermediate plate 24 and rocking platform 18 to align in a substantially flush/parallel configuration.

Before use, and in a static, substantially upright position, a tray 20 is secured to the intermediate platform 24 by placing the tray 20 over the intermediate platform 24 and sliding it down the platform 24. When fully secured, electrical/communication connections in the tray 20 and intermediate platform (34, 36 respectively) will be operatively mated. A vessel/bag (not shown) may then be secured to the tray 20 via one or more attachment mechanisms 38, which in an embodiment are selectively lockable clips located at opposite ends of the tray.

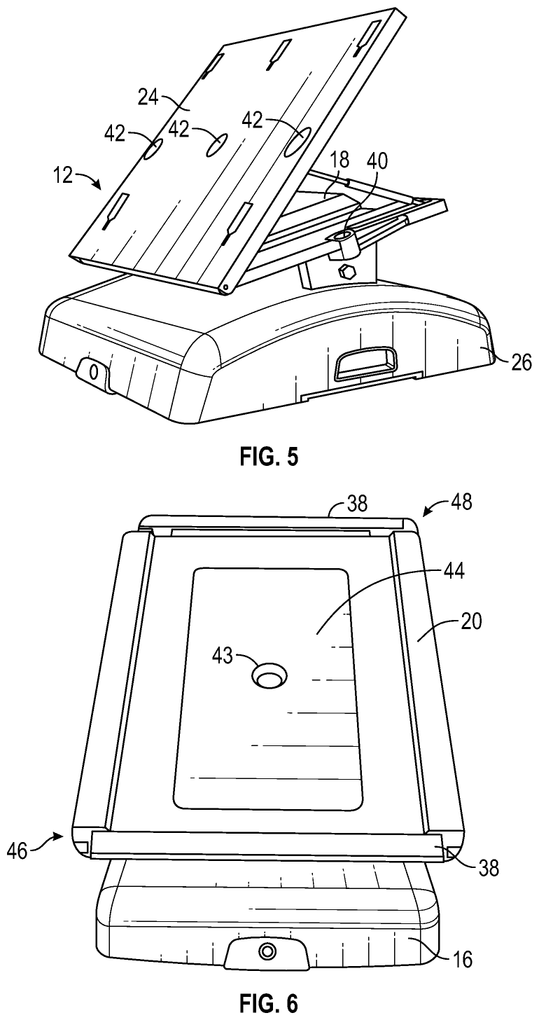

Turning now to FIGS. 4 and 5, the rocking platform 18 has a series of temperature sensors 40, which, in an embodiment is an array of three sensors 40 that are located across an approximate mid-line L of the platform 18. The intermediate platform 24 has apertures 42 which align with the sensors 40 and are sized and shaped to expose the temperature sensors 40 so that they may measure vessel/bag temperature through the tray 20.

Referring to FIG. 6, an existing tray 20 is depicted. As shown, the tray 20 has a tray aperture 43 that allows one of the temperature sensors 40 located on the platform 18 to measure the temperature of fluid in a vessel/bag attached to the tray 20. While FIG. 6 depicts a tray 20 with a single tray aperture 42, other, larger trays that are not depicted feature multiple tray apertures 43 to allow for sensing of multiple/larger reactor bags. In other words, the reactor 12 may be used with a range of vessel/bag sizes through the attachment of larger, e.g., wider, trays.

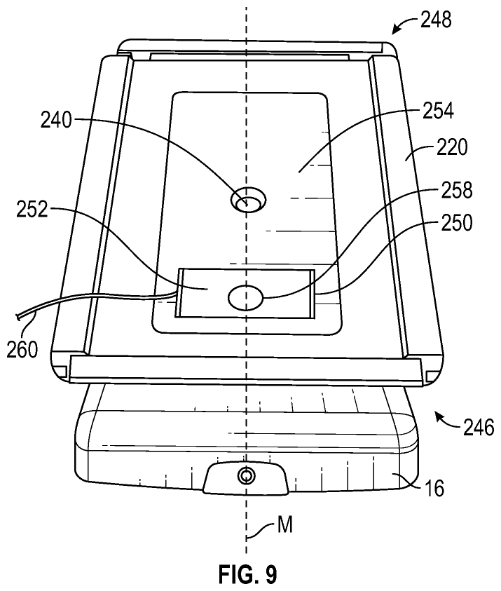

The tray 20 also has an electric heat plate 44 which extends from a lower or first end 46 of the tray 20 to an upper or second end 48. As mentioned, with known rocker bioreactors such as the reactor 12 depicted herein, the heat plate 44 is inoperative when the tray 20 is in its substantially upright sampling/installation position (FIGS. 2 and 9).

Moreover, when substantially upright and fluid in the vessel/bag is proximate the lower, first end 46 of the tray 20, accurate temperature sensing is not possible as the temperature sensors 40 are all located at the midline L of the platform 18.

For at least these reasons, known rocker bioreactors have relatively high minimum working volumes and are not suitable for working with media containing low starting cell numbers/low seeding volumes. As a result, known rocker bioreactors are suitable primarily for cellular growth/expansion such as, for example, production of T-lymphocytes, perfusion culture of T-lymphocytes, and perfusion culture of human natural killer (NK) cells.

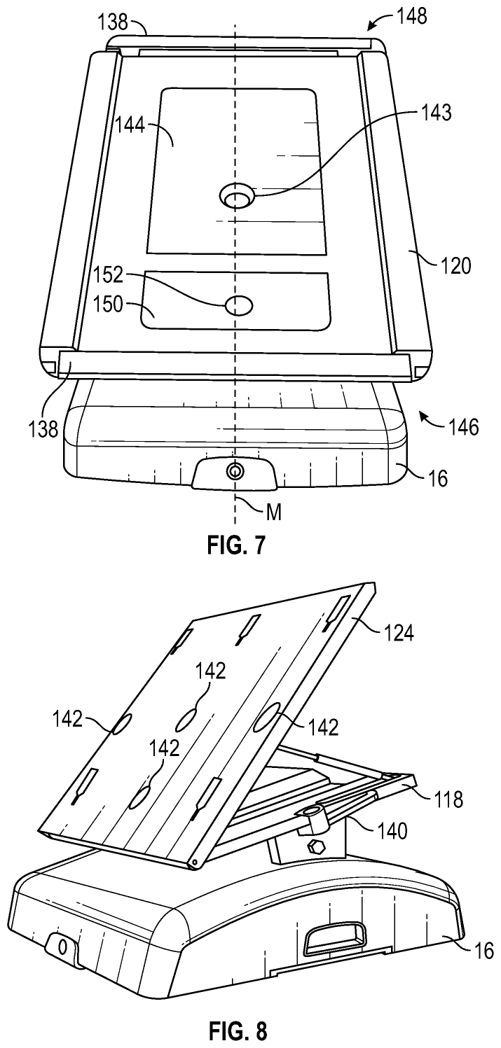

Turning now to FIG. 7 an apparatus for heating fluid in a vessel, e.g., a tray 120, according to an embodiment of the invention is depicted. In embodiments, the tray 120 is configured for use with the rocker bioreactor depicted in FIGS. 1-6 and described above, e.g., a bioreactor vessel/bag may be removably secured to the tray 120 via attachment mechanisms/clips 138. As shown, the tray 120 includes or provides access to two temperature sensors, a first temperature sensor 152 that is located proximate to a lower or first end 146 of the tray 120, and a second temperature sensor 143 that is located between the first temperature sensor and the second end 148 of the tray 120. In embodiments, the second temperature sensor 143 is located at an approximate longitudinal mid-line L of the tray 120 (FIG. 4).

The first and second temperature sensors 152, 143 may be integrated into the tray 120 itself, or they may be configured as described in connection with the tray 20 depicted in FIGS. 5 and 6, and as shown in FIG. 8. Referring specifically to FIG. 8, the first temperature sensor (and second) may be located on or incorporated into the rocking platform 118 of the reactor and may extend through apertures 142 the intermediate plate 124 to access/contact the bag for measurement. As will be appreciated, in such embodiments, the tray 120 will include tray apertures (such as, for example, aperture 46 from FIG. 6) which allow the sensors 140 to extend through and access/contact the bag.

In embodiments where the first temperature sensor 152 (and/or the second sensor 143) is integrated into the tray 120 itself, a variety of temperature sensors may be employed, including ultrasonic sensors. Sensors that sit substantially flush with or slightly below the surface of the tray 120 may be particularly suitable. In embodiments, the first temperature sensor 152 may sit in a recess formed or molded into the tray 120 and may be monitored/controlled via existing electrical/communication connections with the intermediate platform (34, 36), as exemplified by FIG. 3.

In view of the above, the first and second sensors 152, 143 depicted in FIG. 7 are meant to illustrate both sensors embedded into the tray 120 and those located on a rocking platform of the reactor that extend through sensor apertures at the depicted sensor 152, 143 locations.

As mentioned, the first sensor 152 is located proximate the first end 146 of the tray 120. In embodiments, a center of the first sensor 152 is located approximately 6 inches from the bottom of the tray 146, and approximately 5 inches down from a center of the second sensor 143, and at an approximate lateral midline M of the tray 120. In a specific embodiment, the first sensor 152 has an outer diameter of 1.5 inches and an inner diameter of 0.75 inches. That said, in other embodiments, the size and location of the first temperature sensor 152 may depart from the above, so long as the intended functionality is maintained.

Referring again to FIG. 7, embodiments of the tray 120 further include a segmented heating plate. That is, the tray 120 includes two independent heating elements: a first heating element 150, and a second heating element 144. The heating elements 150, 144 may be electric heating plates that are independently operable. In particular, when the reactor is used for, for example, cellular expansion, or another bioprocess that requires rocking/waves, the second heating element 144 may be utilized to heat the contents of the bag, with the first heating element 150 inactive. In certain embodiments, both the first and second heating elements 150, 144 may be activated/utilized during a rocking bioprocessing procedure.

The segmented heating plate also allows for selective heating via the first heating element 150 when the rocking platform of the reactor is in its static, substantially upright position. Given the first heating element's 150 location proximate the first end 146 of the tray 120, in this position, fluid in the bag is gravity driven to collect over the first heating element 150 (and first temperature sensor 152). In this position, heat may be applied to facilitate use of the reactor for bioprocessing procedures (or heated sampling of fluid in the vessel) that do not require rocking. In certain embodiments, the first heating element 150 is configured to provide heat to control temperature of a fluid in a vessel in a dynamic range of approximately 10′ C to about 40° C. As will be appreciated, the temperature sensor 152 is configured to measure fluid temperatures within (and outside) this range.

In embodiments, the first heating element 150 is located approximately 4 inches from the first end 146 of the tray and is approximately 7 inches wide by 3 inches high. As will be appreciated, in other embodiments, the location of size of the first heating element may depart from the above so long as the intended functionality is preserved.

In certain embodiments, the first and second heating elements may be different zones of a single heat plate or may be entirely separate plates. In other embodiments, the tray 120 may include three or more heating zones or elements. The heating elements need not be a specific shape and may be proportioned in a variety of ways without departing from the invention.

As shown, the first temperature sensor 152 is located within/surrounded by the first heating element 150. In certain embodiments, the first heating element 150 and sensor 152 may be separated from one another on the tray 120.

The first and second heating elements 150, 146 may be powered through a variety of means including via the existing electrical/communication connections with the intermediate platform (34, 36), as shown in FIG. 3.

In embodiments, the bioreactor may not include the rocking platform, intermediate plate, tray arrangement as described herein, rather the tray 120 may be directly connected to a rocking mechanism, so long as it is capable of tilting to a substantially upright position. Likewise, the tray 120 need not be removable and, in embodiments, the tray may be/function as the rocking platform. That is, the reactor may utilize a single vessel receiving platform, e.g., a tray, that is connected directly to the rocking mechanism (e.g., motor).

Referring now to FIG. 9, an alternative embodiment of an apparatus for heating fluid in a vessel is depicted. In this embodiment, the apparatus 250 is portable and may be selective added to an existing tray 220. As shown the apparatus 250 includes a heating element 252 (i.e., a “first heating element” when paired with the existing “second heating element” 254 of the tray) and a temperature sensor 258, (the “first temperature sensor” when paired with the existing “second temperature sensor” 240).

In these embodiments, the apparatus 250 may be secured to the tray via an attachment mechanism (not shown), which may include clamps, conventional fasteners, hook and loop fasteners, and other mechanisms that will provide sufficient resistance to movement of the apparatus 250 during and after rocking and non-rocking bioprocessing procedures.

The apparatus 250 may be powered and controlled via one or more external cables 260. In certain embodiments, the first temperature sensor 258 and/or the first heating element 252 may be wirelessly controlled. The first temperature sensor 258 may be a variety of sensor types including ultrasonic sensors. The first heating element 252 is contemplated as an electrically powered metal plate, though use of other heating means may be possible.

The apparatus 250 may have a predetermined location that is proximate the lower, first end 246 of the tray 220, which is opposite the second end 248, and at a lateral midline M. In certain embodiments, the user may be able to select/vary the location of the apparatus 250 on the tray 220 based on the starting cell numbers/seeding volumes, volume of fluid to be processed, and/or type of procedure to be run in the reactor, e.g., the apparatus 250 may be placed closer to or farther from the first end 246.

As stated, embodiments of the invention allow a rocker bioreactor that is capable of both rocking and holding a static, substantially upright vessel attachment/sampling position to run multiple bioprocessing procedures, a feature that is not possible with known reactors. Indeed, embodiments of the invention are drawn to bioprocessing methods that utilize this functionality, as described below.

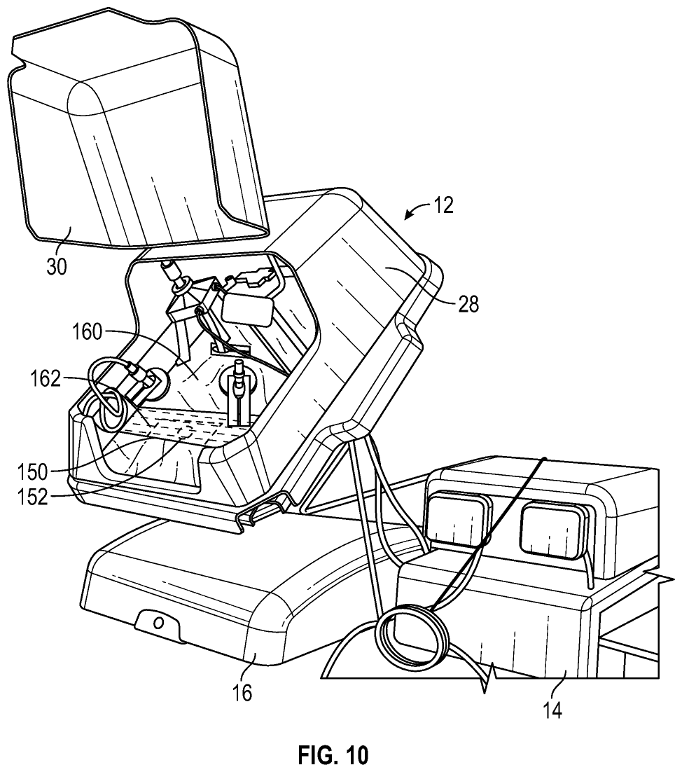

In an embodiment, a user will first secure a vessel, e.g., a bag, containing a fluid to be processed to a platform capable of pivoting about a first axis to create a rocking motion, e.g., a platform of a rocker bioreactor. The fluid in the vessel may then be heated while the platform is in a static, substantially upright position to facilitate a first bioprocessing procedure within the vessel. This position (and process) is depicted in FIG. 10, which shows a bioreactor 12 with a bag 160 and fluid 162. The fluid 162 is heated via the first heating element 150 and temperature may be obtained via the first temperature sensor 152.

After the first bioprocessing procedure is complete, the platform may be pivoted about a second axis/pivot point to a substantially horizontal position where the platform and vessel can then be pivoted about the first axis/pivot point to create a rocking motion to facilitate a second bioprocessing procedure within the vessel. During the second bioprocessing procedure, the fluid 162 may be heated via the second heating element 144 and fluid temperature obtained using the second temperature sensor 143 (FIG. 7).

As mentioned, the present invention allows a rocker bioreactor to perform at least first and second bioprocessing procedures. In embodiments, the first bioprocessing procedure is cellular activation and, in particular, activation in a 2L bioreactor bag with a cell suspension volume of <300 mL. The first bioprocessing procedure is not limited to activation, however, and can include any procedure where rocking is not required yet heat and/or temperature sensing are required or desirable. In embodiments, the first bioprocessing procedure can include heated fluid sampling.

The second bioprocessing procedure is generally one in which a rocking motion is necessary, e.g., cellular expansion. That said, the second procedure is not limited to expansion, and other procedures where fluid may be processed in a substantially horizontal position may be executed without departing from the scope of the invention.

Embodiments contemplate the first and second bioprocessing procedures being executed on the same fluid/cells in a single vessel. However, the first and second bioprocessing procedures may use separate vessels and may not involve processing a single batch of cells.

While embodiments are configured and depicted for use with a 2L bioreactor vessel/bag, embodiments may be utilized with larger vessel/bags. The invention is not limited in this respect.

While in embodiments, a controller, e.g., computer with a processor, executing instructions in a software application, may perform the methods/processes described herein, hard-wired circuitry may be used in place of, or in combination with, software instructions for implementation of the methods/processes of the present invention. Therefore, embodiments of the present invention are not limited to any specific controller or combination of hardware and/or software.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A bioprocessing method comprising:
securing a vessel containing a fluid to a platform capable of pivoting about a first axis;
heating the fluid in the vessel while the platform is in a static, substantially upright position to facilitate a first bioprocessing procedure within the vessel; and
pivoting the platform and vessel about the first axis to create a rocking motion to facilitate a second bioprocessing procedure within the vessel.

2. The method of claim 1, wherein the platform is pivotable about a second axis to and from the substantially upright position; and
wherein the step of pivoting the platform and vessel about the first axis to create a rocking motion to facilitate a second bioprocessing procedure within the vessel occurs after the platform is pivoted about the second axis from the substantially upright position into a substantially horizontal position.

3. The method of claim 1, wherein the vessel is secured to a tray that is removably attached to the platform.

4. The method of claim 3, wherein the tray has a first end and a second end and a first heating element and a first temperature sensor that are proximate the first end.

5. The method of claim 4, wherein the tray further comprises:
a second heating element located between the first heating element and the second end of the tray.

6. The method of claim 4, further comprising:
sensing a temperature of the fluid in connection with the first and/or second bioprocessing procedures.

7. The method of claim 1, further comprising:
heating the fluid in the vessel during the second bioprocessing procedure.

8. The method of claim 1, further comprising:
sampling the fluid in the vessel while it is being heated in the substantially upright position.

9. The method of claim 1, wherein the first bioprocessing procedure is cell activation.

10. The method of claim 9, wherein the cell activation is accomplished with a cell suspension volume of <300 mL.

11. The method of claim 1, wherein the second bioprocessing procedure is cell expansion.

12. The method of claim 1, wherein the platform is at an angle of about 72 degrees from horizontal in the substantially upright position.

13. The method of claim 1, wherein the vessel is a bioreactor bag.

14. The method of claim 13, wherein the bioreactor bag is a 2L bioreactor bag.

15. A system for processing fluid in a vessel, the system comprising:
a tray for removable attachment to a platform that is capable of pivoting about a first axis, the tray having a first end and a second end;
a first heating element and a first temperature sensor that are proximate to the first end of the tray;
an attachment mechanism for securing the vessel to the tray; and
a controller configured for heating the fluid in the vessel while the platform is in a static, substantially upright position to facilitate a first bioprocessing procedure within the vessel and for pivoting the platform and vessel about the first axis to create a rocking motion to facilitate a second bioprocessing procedure within the vessel.

16. The system of claim 15, wherein the platform is pivotable about a second axis to and from the substantially upright position; and
wherein the controller is configured to pivot the platform and vessel about the first axis to create a rocking motion to facilitate a second bioprocessing procedure within the vessel occurs after the platform is pivoted about the second axis from the substantially upright position into a substantially horizontal position.

17. The system of claim 15, further comprising:
a second heating element located between the first heating element and the second end of the tray.

18. The system of claim 15, wherein the first bioprocessing procedure is cell activation.

19. The system of claim 15, wherein the second bioprocessing procedure is cell expansion.

20. The system of claim 15, wherein the platform is at an angle of about 72 degrees from horizontal in the substantially upright position.

21. The system of claim 15, wherein the vessel is a bioreactor bag.

22. The system of claim 21, wherein the bioreactor bag is a 2L bioreactor bag.

23. An apparatus for heating fluid in a vessel, the apparatus comprising:
a first heating element and a first temperature sensor; and
a mechanism for attaching the apparatus to a platform that is capable of pivoting about a first axis, the platform having a first end and a second end; and
wherein the apparatus is configured for selective attachment at a location proximate to the first end of the platform; and
wherein the apparatus is configured for heating the fluid in the vessel while the platform is in a static, substantially upright position during a first bioprocessing procedure within the vessel, and for heating the fluid while the platform is pivoting about the first axis during a second bioprocessing procedure.

24. The apparatus of claim 23, wherein the platform is pivotable about a second axis to and from the substantially upright position; and
wherein the second bioprocessing procedure within the vessel occurs after the platform has pivoted about the second axis from the substantially upright position into a substantially horizontal position.

25. The apparatus of claim 23, wherein the first bioprocessing procedure is cell activation.

26. The apparatus of claim 23, wherein the second bioprocessing procedure is cell expansion.

27. The apparatus of claim 23, wherein the vessel is a 2L bioreactor bag.
